(19) United States
(12) Patent Application Publication
(10) Pub. No.: US 2023/0355108 A1
(43) Pub. Date: Nov. 9, 2023

(54) MONITORING OF BLOOD SUPPLY TO BRAIN

(71) Applicant: Stroke Alert Ltd, Aderet (IL)

(72) Inventors: Hilla BEN-PAZI, Aderet (IL); Revital PERY SHECHTER, Rishon-LeZion (IL); Avshalom MIZRAHI, Zichron-Yaacov (IL)

(73) Assignee: Stroke Alert Ltd, Aderet (IL)

(21) Appl. No.: 18/026,355

(22) PCT Filed: Sep. 14, 2021
(86) PCT No.: PCT/IL2021/051122

§ 371 Date: Mar. 15, 2023

Related U.S. Application Data
(60) Provisional application No. 63/078,602, filed on Sep. 15, 2020.

(51) Int. Cl. A61B 5/02; A61B 5/00

(52) U.S. Cl. CPC A61B 5/02028; A61B 5/02007; A61B 5/4064; A61B 5/6822; A61B 2562/046

(57) ABSTRACT

Systems and methods for monitoring blood supply to the brain of an individual are presented; the system comprising a sensing unit configured to be placed in a vicinity of at least one blood vessel and operable to collect over time, from the at least one blood vessel, measured data indicative of one or more hemodynamic parameters; and a control and processing unit in communication with the sensing unit, the control and processing unit being operable to receive and analyze said measured data, determine hemodynamic data comprising said one or more hemodynamic parameters, analyze said hemodynamic data and, upon detecting a predetermined change over time in the hemodynamic data, generate output data indicative of a blood supply condition to the brain of the individual.

### TECHNOLOGICAL FIELD

The present invention is in the medical field; more specifically, the invention relates to medical devices and methods for monitoring of hemodynamic parameters of a subject.

### BACKGROUND

Adequate blood supply to the brain is crucial to ensure regular brain activity. One medical condition that influences the adequate blood supply to the brain is brain stroke occurrence. Other medical conditions may be related to blood pressure which may affect the blood perfusion to the brain.

Brain stroke presents a possible abrupt deterioration in motor and/or cognitive function, the leading cause of disability. It is the second leading cause of death for people over 60. Over six million people die annually from a stroke. Stroke is usually classified as ischemic or hemorrhagic. The majority of strokes are ischemic. Timely intervention with thrombolysis cures ischemic stroke. It prevents irreversible neuronal death by effective revascularization. Treatment is only effective within the first hours. If left untreated, the patient suffers from a devastating outcome, lifelong disability, and/or death, whereas timely detection and treatment can improve quality of life.

The risk of stroke is exceptionally high around invasive procedures, reaching 10% in cardiac procedures and varies in other types of surgeries, between 0.05-4.4%. Morbidity and mortality are high in the perioperative arena. Generally, strokes cannot be detected in a sedated/sleeping person due to a lack of clinical presenting symptoms, such as lack of motor and cognitive function. As perioperative stroke is ‘silent’, it cannot be treated appropriately and timely. If detected during the operation or immediately after, the patient can be treated. Upon diagnosis, intravenous and endovascular thrombolysis/therapies are viable options.

Approximately 4% to 17% of all strokes occur during hospitalization. Perioperative strokes occur in up to 10% of surgeries, depending on the type of surgery. Another cause of stroke during hospitalizations is in the ICU. For example, a stroke occurs in 4% of patients with non-neurological conditions admitted to the intensive care unit with different risk factors from the general population. Currently, only overt strokes can be treated if recognized on time. Once a stroke occurs in a sedated person in the OR/ICU, it is usually unrecognized on time; thus, it is untreated. As clinical changes obviously cannot be detected in a sedated person, they cannot be treated appropriately once a stroke occurs in this situation. If detected during sedation, the patient can be treated.

Individual factors may also contribute to stroke risks. Age, diabetes, and hypertension increase the individual's odds further. With the aging population and expanding surgical services, these numbers will rise.

### GENERAL DESCRIPTION

The present invention provides a novel approach for monitoring blood supply to the brain. The present invention provides systems, devices, and methods for alerting about the possibility of imminent brain stroke occurrence or about the insufficient blood supply to the brain, which affects brain perfusion, thereby enabling early intervention to keep quality of life or save lives. The novel systems provided in the present invention are robust yet friendly and cost-effective and may be specifically designed for home use by individuals.

In some embodiments, the invention will alert about the possibility of stroke, enabling timely diagnosis and intervention. As reported in the literature, the invention is based, inter alia, on detecting hemodynamic changes indicating a stroke. In some embodiments, the invention can identify hemodynamic features/changes that indicate a stroke or a condition of blood supply to the brain.

According to some aspects of the invention, analysis of one or more hemodynamic parameters measured directly on the user's body by one or more sensors or indirectly obtained from the measurements by the sensor(s) provides an indication about the blood supply to the brain, e.g., indication about stroke occurrence or insufficient perfusion. In some embodiments, the sensor(s) is(are) located in the vicinity of at least one blood vessel (an artery or vein) in the body. In some embodiments, the measurements (indicative of hemodynamic parameters) are obtained from the carotid artery(ies) located on the neck's side(s). In some embodiments, the measurements are obtained from the common and external carotid arteries located on one side of the neck. In some embodiments, the measurements are obtained from the internal and external carotid arteries located on one side of the neck. In some embodiments, the measurements are obtained from the two common carotid arteries located on both sides of the neck. In some embodiments, the measurements are obtained from the Jugular vein(s) located on the side(s) of the neck. In some embodiments, the measurements are obtained from an ECG signal or as an optical pulse measurement.

A sensing unit including at least one sensor may be used for the measurements while located in the vicinity of the monitored blood vessel. For example, at least one sensor may be comfortably attached to the side(s) of the neck to monitor a carotid artery. In one example, at least one sensor is positioned in the vicinity but not attached to the body (e.g., the neck), such as an optical sensor (both transmitting light towards the blood vessel and receiving light that interacts with the blood vessel). The sensor(s) utilize(s) one or more modalities, individually or collectively, sequentially or simultaneously, to provide the measurements of the hemodynamic parameters, such as optical, ultrasound-based (e.g., Doppler), and electrical (e.g., capacitive, ECG) measurement modalities. The analysis of the measurements points out an occurring/imminent stroke or another condition of the change in blood supply to the brain.

In some embodiments, the analysis includes comparing the measured hemodynamic parameter(s) to a corresponding hemodynamic history data. The hemodynamic history data may include baseline data possibly obtained prior to collecting the data indicative of the occurring/imminent stroke. The baseline data may include personal baseline data obtained from the same monitored individual or collective baseline data obtained from a plurality of previously monitored individuals and saved in an accessible database.

In some embodiments, Artificial Intelligence and/or Machine Learning may be applied to the hemodynamic history/baseline data accumulated over a predetermined time period from individual(s) using the principles of the present invention to generate dynamic reference hemodynamic data that forms at least part of the hemodynamic history data to which the personal measured data is compared.

The hemodynamic history data may include other quantitative data that indicate a possibility of a stroke, e.g., quantitative data that has been recognized by the medical field as indicating a stroke occurrence, possibly based on medical research (for example, a unilateral decrease of over 20% for more than 30 seconds).

In some embodiments, the analysis relates to hemodynamic data measured/obtained from at least one sensor. In the case of one sensor, the hemodynamic data is collected from a specific blood vessel. In another embodiment, two or more sensors are used. In some embodiments, the two measurements are extracted from the same side of the neck. In some embodiments, the two measurements are extracted from two sides of the neck. In some embodiments, the measured data obtained from a blood vessel is compared to the previous data of that blood vessel. In some embodiments, the measured data obtained from each blood vessel is alternatively or additionally compared to measured data obtained from other blood vessels (s). For example, the analysis includes a comparison between the characteristics (e.g., values) of the hemodynamic parameter(s) obtained from the two common carotid arteries on both sides of the neck.

Thus, according to a first aspect of the invention, there is provided a system for monitoring of blood supply to a brain of an individual, the system comprising:

a sensing unit configured and operable to be placed in a vicinity of at least one blood vessel and collect over time, from at least one blood vessel, measured data indicative of one or more hemodynamic parameters; and

A control and processing unit in communication with the sensing unit, the control, and processing unit is configured and operable to receive and analyze said measured data, determine hemodynamic data comprising said one or more hemodynamic parameters, analyze said hemodynamic data, and upon detecting a predetermined change over time generate output data indicative of a blood supply condition to the brain of the individual. In some embodiments of the invention, the monitoring system detects local changes in blood flow to the brain, optionally correlated with various physiological parameters to provide stroke alerts. At least a part of the monitoring system may be configured as a bedside device or a wearable device.

In some embodiments, hemodynamic changes are detected with a local system that includes at least two sensors, with at least one sensor for each carotid artery and/or Jugular vein on the right and left sides, and a controller designed to control, process, analyze and send alerts. In some embodiments, stroke detection involves acute arterial and/or venous hemodynamic changes compared to a baseline, especially the variation in these changes between the sensors.

In some embodiments of the invention, the monitoring system detects unique systemic hemodynamic changes, such as heart rate variability, to provide stroke alerts.

In some embodiments, the analysis of the hemodynamic changes is combined with other physiologic parameters to increase sensitivity and specificity in detecting the danger of stroke.

In some embodiments, the control and processing unit is configured and operable to analyze the first portion of said measured data collected over the first time period, determine a corresponding first portion of the hemodynamic data and save the first portion of the hemodynamic data as a baseline data, analyze the second portion of said measured data continuously collected over the second time period and determine a corresponding second portion of the hemodynamic data, apply a comparison between the second portion of the hemodynamic data and a history data comprising the baseline data, to generate said output data indicative of a blood supply condition to the brain of the individual. The history data may comprise baseline and/or event data of one or more monitored individuals. The event data can be, for example, historical data relaying the expected changes in hemodynamic parameters indicating the occurrence of stroke.

In some embodiments, the sensing unit is configured and operable to be placed in a vicinity of at least two blood vessels (e.g., carotid arteries) located respectively on the right and left sides of a neck of the individual, and collect the measured data from the at least two blood vessels, the measured data thereby comprising right and left measured data.

In some embodiments, the control and processing unit is configured and operable to analyze the right and left measured data and determine the hemodynamic data comprising right and left hemodynamic data for the blood vessels (e.g., carotid arteries) on the right and left sides, respectively, and use the right and left hemodynamic data to generate said output data indicative of a blood supply condition to the brain of the individual.

In some embodiments, the control and processing unit is configured and operable to apply a comparison between the right and left hemodynamic data to generate said output data indicative of a blood supply condition to the individual's brain.

In some embodiments, the control and processing unit is configured and operable to apply, for each one of the right and left sides, a comparison between the hemodynamic data and a respective baseline data determined based on a first portion of the hemodynamic data collected over the first period, to generate said output data indicative of a blood supply condition to the brain of the individual.

In some embodiments, the control and processing unit is configured and operable to analyze systemic hemodynamic changes from baseline and apply a comparison between the hemodynamic history data and the newly acquired hemodynamic data to generate said output data indicative of a blood supply condition to the brain of the individual.

In some embodiments, the control and processing unit is wirelessly connected to the said sensing unit.

In some embodiments, the sensing unit and said control and processing unit are located on a common platform.

In some embodiments, the sensing unit comprises an array of sensors configured and operable to collect the measured data from the blood vessel (e.g., carotid artery) along with a predetermined distance thereof. In some embodiments, the sensing unit comprises a two-dimensional array of sensors configured to be placed in a vicinity of an area of the individual's neck covering said blood vessel (e.g., carotid artery), said control and processing unit is configured and operable to activate one or more sensors of the two-dimensional array of sensors to collect the measured data.

In some embodiments, the sensing unit comprised a capacitive sensor, and said hemodynamic data comprises one or more of the following: blood vessel expansion data and blood flow data.

In some embodiments, the sensing unit comprises a sensor (pulse, ECG) configured and operable to collect the measured data over time.

In some embodiments, the sensing unit comprises an ultrasound sensor, and said emodynamic data comprises blood flow data.

In some embodiments, the sensing unit comprises a piezoelectric sensor.

In some embodiments, the sensing unit comprises a resistive/optical strain gauge sensor.

In some embodiments, the sensing unit comprises an imaging-based sensor that can capture a moving image related to blood flow.

In some embodiments, where the sensor unit is optically based, whether a single sensor, array of sensors, or imaging sensor, the light can be transmitted and/or received via optical fibers.

In some embodiments, at least one blood vessel is one or more of the following: common carotid artery, external carotid artery, internal carotid artery, and jugular vein.

In some embodiments, the control and processing unit is configured and operable to generate the output data being indicative of an increase or a decrease of blood supply to the brain.

In some embodiments, the control and processing unit is configured and operable to generate the output data being indicative of a stroke occurring in the individual's brain.

In some embodiments, the control and processing unit is configured and operable to generate the output data being indicative of an increase or a decrease of hemodynamic parameters (such as heart rate variability), indicative of a stroke.

In some embodiments, the control and processing unit is configured and operable to receive and analyze medical data in addition to the hemodynamic data to generate thereby the output data indicative of a blood supply condition to the brain of the individual.

In some embodiments, the medical data comprises one or more of the following: EEG data, ECG data, pulse data, Emboli data, and carotid artery and/or jugular vein hemodynamic data.

According to another aspect of the invention, there is provided a method for determining a blood supply to brain condition, the method comprising:
receiving measured data collected over time from at least one blood vessel;
analyzing the measured data and determining hemodynamic data comprising one or more hemodynamic parameters;
analyzing the hemodynamic data and upon detecting a predetermined change in one or more hemodynamic parameters determining the condition of the blood supply to the brain.

In some embodiments, the measured data comprises first and second measured data collected respectively over first and second periods, said analyzing of the measured data comprising determining first and second hemodynamic data respectively, and said analyzing of the hemodynamic data comprising comparing the second hemodynamic data with the first hemodynamic data for detecting the predetermined change in the one or more hemodynamic parameters and determining the condition of blood supply to the brain. In some embodiments, the first hemodynamic data forms baseline data saved and used for comparing with hemodynamic data collected later on.

In some embodiments, analyzing the hemodynamic data comprises comparing the hemodynamic data to baseline and/or event data comprising hemodynamic data of one or more individuals who have been monitored.

In some embodiments, the measured data is collected from at least two blood vessels located respectively on the right and left sides of a neck of the individual, the measured data thereby comprising right and left measured data respectively, and the hemodynamic data comprising right and left hemodynamic data respectively.

In some embodiments, the analysis of the hemodynamic data comprises analyzing each of the right and left hemodynamic data and applying a comparison between the respective hemodynamic data and a respective baseline data determined based on a first portion of the respective hemodynamic data to determine the blood supply condition to the brain of the individual.

In some embodiments, the analysis of the hemodynamic data comprises applying a comparison between the right and left hemodynamic data to determine the blood supply condition to the individual's brain.

In some embodiments, the hemodynamic data comprises one or more of the following: blood vessel's expansion data and blood flow data.

In some embodiments, at least one blood vessel is one or more of the following: common carotid artery, external carotid artery, internal carotid artery, and jugular vein.

In some embodiments, the blood supply to brain condition is indicative of a stroke occurring in the individual's brain.

In some embodiments, the blood supply to brain condition is indicative of an increase or a decrease of blood supply to the brain.

In some embodiments, the method comprises receiving and analyzing medical data in addition to the hemodynamic data for determining the blood supply condition to the brain of the individual. The medical data may comprise one or more of the following: EEG data, Emboli data, ECG data, Heart Rate data, and carotid artery and/or jugular vein hemodynamic data.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates, in the way of a block diagram, a non-limiting example of a system for monitoring blood supply parameters to an individual's brain, according to some embodiments of the invention;

FIG. 2 illustrates, in the way of a block diagram, a non-limiting example of a system for monitoring blood supply parameters to an individual's brain utilizing baseline data, according to some embodiments of the invention;

FIG. 3 illustrates a non-limiting example of a sensing unit configured for providing blood flow/velocity measurements;

FIG. 4 illustrates a non-limiting example of a sensing unit including a two-dimensional sensor array;

FIG. 5 illustrates, in the way of a flow diagram, a non-limiting example of a process/method for determining a condition of blood supply to the brain.

### DETAILED DESCRIPTION OF EMBODIMENTS

In the following, non-limiting examples of systems, devices, and methods in accordance with the invention are described.

In one specific non-limiting example, the invention is described as being applied to the carotid arteries, which may teach about hemodynamic parameters values or hemodynamic changes relevant to the blood supply to the brain.

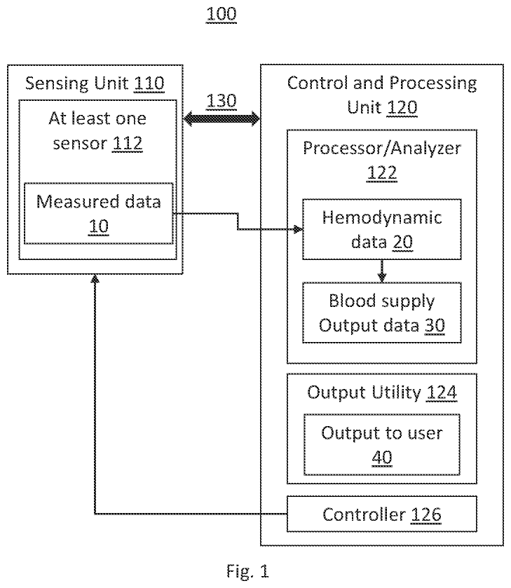

Reference is made to FIG. 1 that illustrates a non-limiting example of a system 100 for monitoring the brain's blood supply conditions/parameters, in accordance with the invention. System 100 includes a sensing unit 110 and a control and processing unit 120 configured to communicate therebetween via a communication assembly/network/protocol 130.

The sensing unit 110 includes at least one sensor 112 configured and operable to collect measured data 10 indicative of one or more hemodynamic parameters of the monitored individual.

The sensing unit 110 is configured and operable to be positioned in the vicinity of at least one blood vessel carrying blood to/from the brain. For example, the sensing unit 110 may be attached to the individual's neck to monitor hemodynamic parameter(s) from the carotid artery(ies). Ascending carotid arteries carrying blood to the brain are located on the right and left sides of the neck. For example, at least one sensor 112 may be attached to the individual's neck in the vicinity of one or more of the following: the common carotid artery, the internal carotid artery (stemming from the common carotid artery and supplying blood to the brain arteries), and/or the external carotid artery (stemming from the common carotid artery and supplying blood to the facial area).

As mentioned above, the sensing unit 110 may include one or more sensors 112, each of the sensors is configured and operable to collect data in one or more measurement modalities (e.g., optical, ultrasound, capacitive). Some non-limiting examples of the sensor(s) 112 are described further below.

The control and processing unit 120 includes a processor/analyzer 122 configured and operable to receive, process, and analyze the measured data 10 and generate output hemodynamic data 20 and output data 30 indicative of the brain blood supply condition, and an output utility 124 configured and operable to generate an output to a user 40 indicative of the blood supply condition, such as an alert to the user. In some embodiments, the control and processing unit 120 includes a controller 126 configured and operable to control the sensing unit 110 and collect the measured data 10. The controller 126 is also configured and operable for providing Quality Assurance that the signal detected by the sensing unit 110 is stable and reliable.

In some embodiments, system 100 is enclosed in a single housing accommodating both the sensing unit 110 and the control and processing unit 120. In some embodiments, the sensing unit 110 and the control and processing unit 120 are accommodated in two different housings. In some embodiments, part of the control and processing unit 120 is located within the same housing as the sensing unit 110 (such as the controller 126), while another part is located in another housing. In some embodiments, the control, and processing unit 120 is distributed between at least two housings/locations (including remote locations such as a remote server/cloud server). In some embodiments, the control, and processing unit 120 is at least partially implemented as a software module in a computing device.

The control and processing unit 120 communicates with the sensing unit 110 via the communication assembly/network/protocol 130, utilizing a communication technique known in the field, either wired or wireless communication, such as Bluetooth or Wi-Fi communication.

Generally, system 100, and specifically the communication assembly 130, may include (not explicitly illustrated in the figures):
A/D converter (may be integral with the sensing unit 110), depending on the transmission protocol for transmitting the signals detected by the sensing unit 110 to the control and processing unit 120. The A/D converter should preferably be of at least 8 bits to meet the specific application requirements.
Bluetooth/Wi-Fi/other transmitters for transmitting the signals to the control and processing unit 120 or a wiring network. The method of transmitting the data, whether wired or wireless, depends on safety, environmental and ergonomic considerations;
A battery may be included for powering the communication assembly and/or the sensing unit. The battery should preferably be small, lightweight, and flat, designed to allow for continuous operation of enough time, for example, at least a few hours; a battery may be included in the control and processing unit;
A receiver (that may form part of the control and processing unit 120), designed to collect signals from the sensing unit 110, allowing for possible synchronous operation when a plurality of sensors are present. In some embodiments, where the monitored hemodynamic parameter is the pulse wave of the blood, the receiver is adapted to a sampling rate of more than 10Hz.

In some embodiments, the processor/analyzer 122 processes/analyzes the measured data 10 received from the sensing unit 110 to determine a hemodynamic parameter 20 such as blood velocity, blood flow, blood volume, heart rate variability, vessel volume, blood characteristics such as oxygen or other constituents. The processor/analyzer 122 then analyzes the hemodynamic parameter(s) 20. Upon detecting a predetermined change over time in the hemodynamic parameter(s), individually or among different parameters, the processor/analyzer 122 generates output data 30 indicative of the blood supply to the brain. Based on the output data 30, the output utility 124 generates a corresponding output to the user, e.g., a perfusion alert or a stroke alert. In some embodiments, the processor/analyzer 122 generates the output data 30 once there is a difference above a predetermined threshold between the measured data 10 or the processed hemodynamic data 20 and baseline data, as will be further described below.

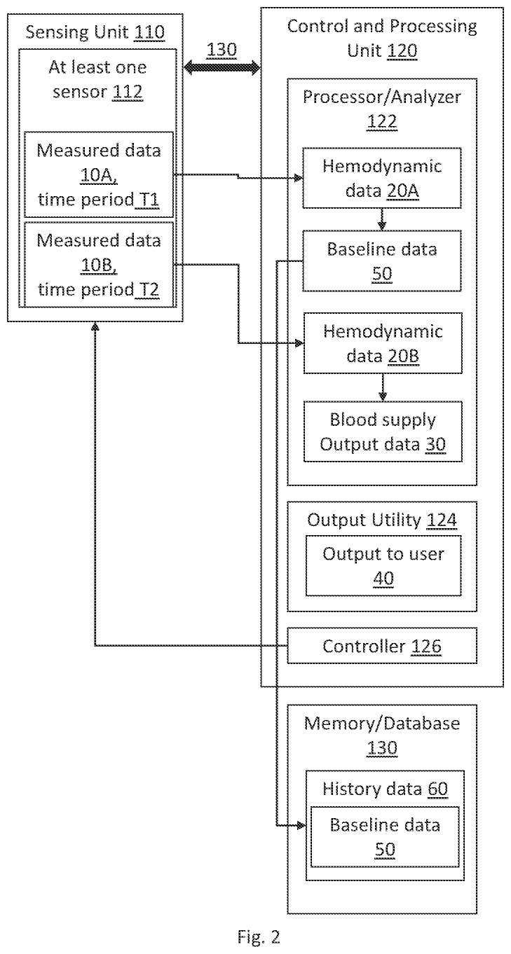

Reference is made to FIG. 2, illustrating a non-limiting example of the analysis of measured data by the control and processing unit 120 for determining the blood supply condition data 30. As shown, the control and processing unit 120 is configured and operable to determine the output data 30 by analyzing a first portion of the measured data 10A collected over the first period T1, determining a corresponding first portion of the hemodynamic data 20A and saving the first portion of the hemodynamic data 20A as a baseline data 50, possibly in a local/distant memory or database 130. The control and processing unit 120 then analyzes a second portion of the measured data 10B continuously collected over the second period T2 and determines a corresponding second portion of the hemodynamic data 20B. The control and processing unit 120 then applies a comparison between the second portion of the hemodynamic data 20B and history data 60 that includes the baseline data 50 and possibly other data as will be further described below and generates the output data 30 indicative of the blood supply condition to the brain of the individual.

It is noted that the baseline line data 50 can be dynamic data that is continuously updated. So, for example, the baseline data can be updated with the hemodynamic data 20B. Further, the baseline data 50 and/or generally the history data 60 can be based on measured data from the same individual or measured data from a plurality of individuals.

Herein, as appreciated, the baseline data 50 typically refers to the characteristic/value of the same hemodynamic parameter in the same blood vessel during a presumed normal/healthy period. Alternatively, the baseline data 50 of the hemodynamic parameter may refer to a relation (e.g., difference, division, etc.) or correlation between the values of the hemodynamic parameter in different blood vessels, such as in the two common carotids. Nevertheless, the baseline data 50 may refer to a relation or correlation between two or more hemodynamic parameters measured in the same blood vessel or different blood vessels. The baseline data 50 may be calculated as a mean value and/or as an average of the hemodynamic parameter's characteristic (e.g., value) monitored during a period prior to the stroke/blood supply condition. As described above, the baseline data 50 may be obtained from the same individual during a predetermined period, and as such forms, personal baseline data, or the baseline data may be collective baseline data obtained from a plurality of individuals and updated with each newly monitored individual including the currently monitored individual. It is appreciated that the history data 60, to which the hemodynamic parameters 20 of the currently monitored individual are compared, may include both the personal and the collective baseline data. Also, it is appreciated that the history data 60 may include hemodynamic data that the medical field has recognized (e.g., through research, applying machine learning algorithms, etc.) as being indicative of a stroke or a condition of the blood supply to the brain.

In one non-limiting example of the invention, the monitored hemodynamic parameter 20 is a parameter of blood propagation (e.g., pulse wave velocity) in the carotid arteries. The main anatomical definitions related to brain stroke location are Ipsilateral—the same side as the stroke and Contralateral—the opposite side of the stroke. If the stroke occurs on the left side of the brain, the left side is the ipsilateral side, and the right side is the contralateral side. At baseline, e.g., in a normal/healthy situation, the flow is more or less symmetrical (there may be constant differences). For example, the flow in the right common carotid (CC) is equal to the flow in the left common carotid, or the flow in the right common carotid has a constant difference (less or more) from the left common carotid. At each side of the neck, most of the blood flows through the larger internal carotid (IC) and less through the external carotid (EC). As a stroke evolves on the left side, the blood flow in the left, ipsilateral, internal carotid decreases compared to the baseline flow in the same internal carotid or the contralateral internal carotid, and the blood flow in the ipsilateral external carotid increases or is kept similar to the baseline flow in the same external carotid or the contralateral external carotid. The blood flow in the left common carotid is lower than the baseline flow in the same common carotid and possibly lower than the blood flow in the right, contralateral, common carotid artery (for example, if at the baseline, the blood flow in the two common carotid arteries is equal). These changes in the hemodynamic parameters and circulation over time occur mainly on the ipsilateral side as opposed to the contralateral side, which mostly remains unchanged as long as the stroke is concerned.

When a stroke occurs, there will be, compared to the baseline, hemodynamic changes that could be detected. Accordingly, in some embodiments of the invention, initial baseline data (e.g., blood flow in a normal/healthy/pre-stroke condition) is obtained for each sole patient and used for comparison with the measured data collected thereafter during a stroke.

Other changes in blood flow may indicate a problem that is not necessarily stroke-related. Bilateral changes from the baseline data, reflected in the two common carotid arteries, may indicate increased/decreased global cerebral perfusion. Moreover, in cases of internal carotid narrowing (e.g., as a result of debris accumulation), the blood flow in the two common carotid arteries may differ with respect to each other (lower flow in the common carotid at the side of the narrowing internal carotid).

Another example is increased flow in both common carotid arteries. This increase could result from other systematic changes, such as administered Intra Venous (IV) fluids.

System 100 may also detect hemodynamic changes in time and may alert for significant specific changes. System 100 can detect these changes, from the baseline, in the hemodynamic parameters, such as the blood flow, and generate a corresponding output data/alert.

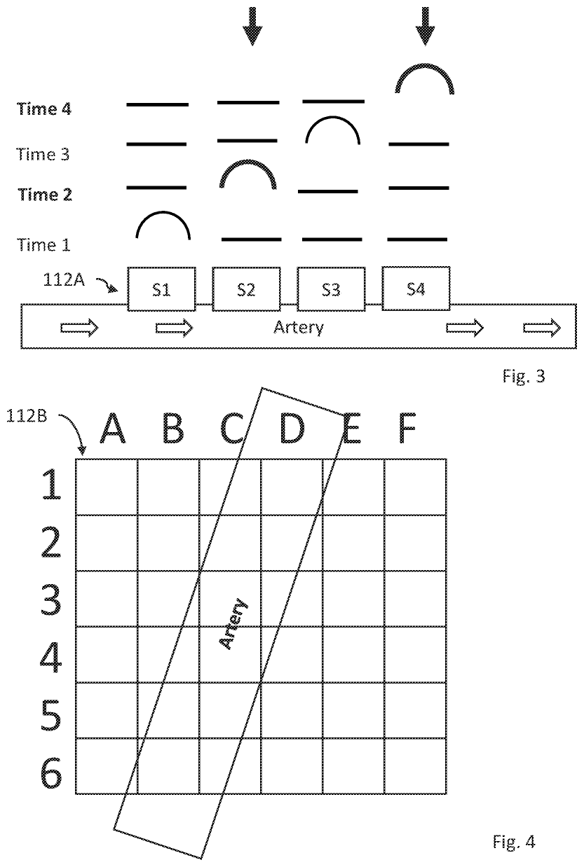

Reference is made to FIG. 3 schematically illustrating a non-limiting example of at least one sensor, which is a hemodynamic sensor/detector.

A sensor array 112A of four sensors S1-S4 (an array of at least two sensors is required) is shown lying along an axis of an artery. Each sensor can record a change over time in at least one parameter related to blood pulse in the artery portion beneath the sensor. In a non-limiting example, such sensors can be capacitance-based sensors detecting the pulsation of the blood flowing in the artery by the change of the artery's diameter as reflected by a change in the skin displacement/movement. The chosen sampling rate enables to capture of the change in the pulse waveform over time, represented as time 1-time 4, such that sensor S1 records the pulse waveform at time 1, sensor S2 records the pulse waveform at time 2, and so on. Each sensor along the way can detect the wave of pulsation of the blood, such that the location of the sensor along the artery and the distances between the sensors can indicate the velocity of the pulse propagation (pulse wave velocity PWV). The PWV is the distance passed over time. In the described example, it can be said that:

PWV=distance/time=distance between any two sensors/time difference of recording the propagating pulse.

Measuring the pulse/waveform characteristics, such as velocity, using an array or a matrix of sensors could be achieved by other sensor types, such as ultrasound sensors detecting the echo (Doppler) from the artery or optical sensors detecting changes in absorption of light as a function of time, or other types as listed further below.

The control and processing unit is operable to analyze the pulse/waveform characteristics, including the shape, area, maximum, minimum, width, and others.

In one non-limiting example, the sensor(s) can be made out of piezoelectric material and operable, when attached to the body surface above the blood vessel of interest, to generate electrical signals as a function of mechanical variations occurring below the sensor(s) as a result of the blood flow. In one non-limiting example, the sensor(s) is(are) made from at least one of the following: Bimorph Ceramic material or Polyvinylidene Fluoride (PVDF).

Each sensor in the sensor array can be separate, or the sensor array can be mounted on a common mechanical support. The latter option is easier for attaching the sensor array to the body. As appreciated, any sensor unit used should preferably be flexible to conform to the body's geometry (e.g., the neck).

The electrical circuit that transmits the electrical signal from the sensing unit to the control and processing unit can be attached directly to the mechanical support of the sensing unit or placed at a distance from the sensor(s) to decrease the size and weight of the sensing unit.

In one non-limiting example, the sensing unit includes resistive/optical strain gauge sensor(s).

In yet another non-limiting example, the sensing unit includes an imaging-based sensor(s), that can capture a moving image related to blood flow.

In some embodiments, where the sensor(s) is(are) optically based, the light can be transmitted thereto and/or received therefrom via optical fibers.

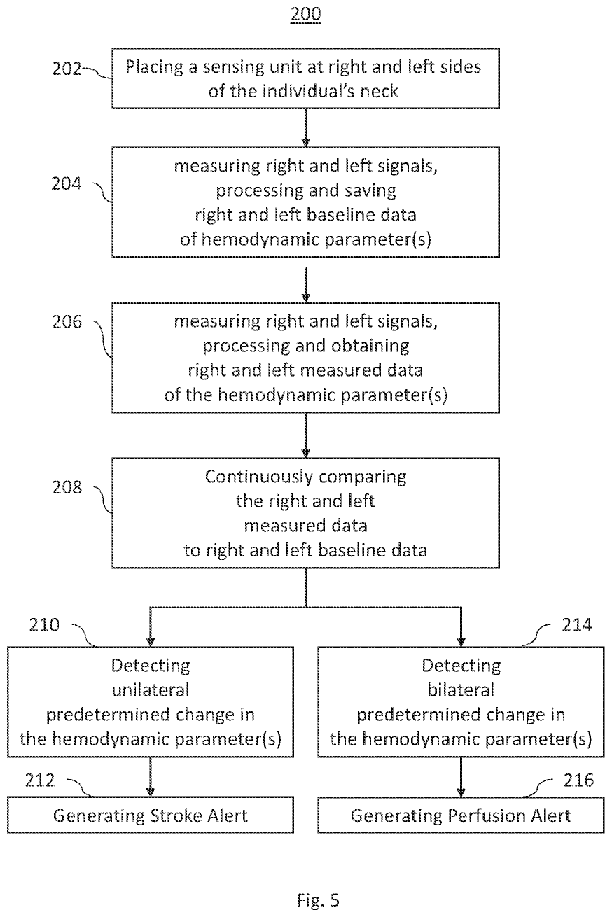

As the itinerary of the artery may not be readily known without imaging, the one-dimensional sensor array exemplified in FIG. 3 may turn out to be limited in providing the required measured data. FIG. 4 illustrates a non-limiting example of a two-dimensional sensor array 112B, including 6∴6=36 sensor elements A1-F6. This matrix configuration allows easy placement of sensor 112B on the body (neck) without knowing the exact artery itinerary to place the sensor accurately above the artery. In this case, at the beginning of monitoring, system 100 collects signals from all the sensors in the matrix, analyzes them, and determines the sensors lying along the path of the monitored artery. In the shown example, signals (e.g., Artery capacitance pulses) can be detected by sensors B5-B6, C1-C6, D1-D2.

A non-limiting example of a process 200 utilized by the system 100 may include the following steps, as illustrated in the flow diagram of FIG. 5. In the figure, two scenarios are described, a first scenario is when a change in the hemodynamic parameter(s) occurs on one side of the neck, and a second scenario is when a change in the hemodynamic parameter(s) occurs on two sides of the neck. It is appreciated that these two scenarios are not co-related.

In step 202, it was selecting the blood vessels to be monitored (e.g., right and left common carotids) and placing corresponding sensing units (each including one or more sensors) in the vicinity. For example, the sensor array described in FIG. 3 or 4 is used to determine the blood flow/velocity (measuring the pulse wave velocity). Also, in the described example, the sensor(s) are placed on both the right and left sides of the individual's neck to collect measurements from the two common carotids at least. It is again noted that in another example (not shown), the sensor(s) are placed on one side of the neck and configured to collect measurements from the right or left common carotid alone, or from any two blood vessels among the common, external and internal carotids on the same side of the neck.

In step 204, right and left signals are measured by the right and left sensing units, processing the signals and saving right and left baseline data (personal baseline) of one or more hemodynamic parameters of the common carotids on both sides (left and right) of the neck. It is assumed that the baseline data refers to the time of a healthy condition of the individual. Typically, this step is performed once at the start of the monitoring process. Possibly, the baseline data is continuously updated by more measurements. Possibly, the baseline data is obtained from measurements obtained on a plurality of individuals, e.g., based on research and depending on the monitored hemodynamic parameter(s).

In step 206, continue collecting continuous measurements indicative of one or more hemodynamic parameters from both right and left common carotids and processing the signals to obtain right and left measured data of the hemodynamic parameter(s).

In step 208, continuously analyzing the measurements by comparing the right and left measured data each to its corresponding baseline data to detect changes from the corresponding baseline data of the individual. Possibly, comparing the continuously received measured data to historical data, including parameters other than the specific hemodynamic parameter(s) being measured. Possibly, the detected changes are above a predetermined threshold.

Analyzing the differences and comparing, using an algorithm based on historical data and baseline data stored, to find a connection/correlation between these types of differences and the blood supply condition.

In step 210, if the detected changes are unilateral (on one side of the neck), generating, at step 212, an alert of the possibility of a stroke at the neck side facing changes.

In step 214, if the detected changes are bilateral happening on both sides, generating, at step 216, an alert of a perfusion problem.

As mentioned, the analysis of the differences may include a comparison to the baseline data (both personal and collective found in a database).

In some embodiments, the system 100 may be configured to detect continuous and/or discrete changes from the baseline.

In some embodiments, as mentioned above, the invention may utilize other inputs of parameters to generate the output data indicative of the blood supply condition to the brain, for example:

Heart Rate Variability;

Emboli detection—Detection of increased particle flow relative to a baseline in terms of (1) size, (2) volume, and (3) frequency;

Venous drainage—As the stroke evolves, there will be a hemodynamic change in the ipsilateral internal jugular vein compared to (1) baseline, (2) contralateral jugular, and (3) ipsilateral external jugular vein. Therefore, measurement of vein hemodynamics may also be included for corroborating stroke detection;

EEG—As one hemisphere suffers ischemia relative to the contralateral hemisphere, changes in physiological parameters, such as EEG, can be detected compared to a predetermined baseline, such as changes in (1) synchrony and (2) symmetry, both in frequency and amplitude. Therefore, the measurement of EEG may also be included for corroborating stroke detection.

Accordingly, secondary measurements corroborating detection can be used together with the hemodynamic measurement. The secondary measurements can be provided by sensors of the system of the invention or by external sensors communicating with the system.

1. A system for monitoring blood supply to the brain of an individual, the system comprising:
a sensing unit configured and operable to be placed in a vicinity of at least one blood vessel and collect over time, from the at least one blood vessel, measured data indicative of one or more hemodynamic parameters; and
a control and processing unit in communication with the sensing unit, the control and processing unit being configured and operable to receive and analyze said measured data, determine hemodynamic data comprising said one or more hemodynamic parameters, analyze said hemodynamic data and upon detecting a predetermined change over time generate output data indicative of a blood supply condition to the brain of the individual.

2. The system according to claim 1, wherein said control and processing unit is configured and operable to analyze a first portion of said measured data collected over a first time period, determine a corresponding first portion of the hemodynamic data and save the first portion of the hemodynamic data as a baseline data, analyze a second portion of said measured data continuously collected over a second time period and determine a corresponding second portion of the hemodynamic data, apply a comparison between the second portion of the hemodynamic data and history data comprising the baseline data to generate said output data indicative of a blood supply condition to the brain of the individual.

3. The system according to claim 2, wherein said history data comprises baseline data of one or more individuals who have been monitored.

4. The system according to claim 1, wherein said sensing unit is configured and operable to be placed in a vicinity of at least two blood vessels located respectively on right and left sides of a neck of the individual, and collect the measured data from the at least two blood vessels, the measured data thereby comprising right and left measured data.

5. The system according to claim 4, wherein said control and processing unit is configured and operable to analyze the right and left measured data and determine the hemodynamic data comprising right and left hemodynamic data for the blood vessels on the right and left sides of the neck, respectively, and use the right and left hemodynamic data to generate said output data indicative of a blood supply condition to the brain of the individual.

6. The system according to claim 5, wherein said control and processing unit is configured and operable to apply, for each one of the right and left sides of the neck, a comparison between the respective hemodynamic data and a respective baseline data determined based on a first portion of the hemodynamic data collected over a first time period, to generate said output data indicative of a blood supply condition to the brain of the individual.

7. The system according to claim 5, wherein said control and processing unit is configured and operable to apply a comparison between the right and left hemodynamic data to generate said output data indicative of a blood supply condition to the brain of the individual.

8-9. (canceled)

10. The system according to claim 1, wherein said sensing unit comprises an array of sensors configured and operable to collect the measured data from the blood vessel along a predetermined distance thereof.

11. The system according to claim 1, wherein said sensing unit comprises a two-dimensional array of sensors configured to be placed in a vicinity of an area of the individual's body covering said blood vessel, said control and processing unit being configured and operable to activate one or more sensors of the two-dimensional array of sensors to collect the measured data.

12. The system according to claim 1, wherein said sensing unit comprises a capacitive sensor and said hemodynamic data comprises one or more of the following: blood vessel's expansion data and blood flow data.

13. The system according to claim 1, wherein said sensing unit comprises an ultrasound sensor and said hemodynamic data comprises blood flow data.

14. The system according to claim 1, wherein said at least one blood vessel is one or more of the following:
common carotid artery, external carotid artery, internal carotid artery and jugular vein.

15. The system according to claim 1, wherein said control and processing unit is configured and operable to generate the output data being indicative of a stroke occurring in the individual's brain.

16. The system according to claim 1, wherein said control and processing unit is configured and operable to generate the output data being indicative of an increase or a decrease of blood supply to the brain.

17. The system according to claim 1, wherein said control and processing unit is configured and operable to receive and analyze medical data in addition to the hemodynamic data to thereby generate the output data indicative of a blood supply condition to the brain of the individual.

18. The system according to claim 17, wherein said medical data comprises one or more of the following: EEG data, Emboli data, ECG data, Heart Rate data, and carotid artery and/or jugular vein hemodynamic data.

19. A method for determining a blood supply condition to brain for an individual, the method comprising:
receiving measured data collected over time from at least one blood vessel;
analyzing the measured data and determining hemodynamic data comprising one or more hemodynamic parameters;
analyzing the hemodynamic data, and upon detecting a predetermined change in the one or more hemodynamic parameters determining the blood supply condition to the brain of the individual.

20. The method according to claim 19, wherein said measured data comprises first and second measured data collected respectively over first and second periods of time, said analyzing of the measured data comprising determining first and second hemodynamic data respectively, and said analyzing of the hemodynamic data comprising comparing the second hemodynamic data with the first hemodynamic data for detecting the predetermined change in the one or more hemodynamic parameters and determining the blood supply condition to the brain of the individual.

21. The method according to claim 19, wherein said analyzing of the hemodynamic data comprises comparing the hemodynamic data to baseline data comprising hemodynamic data of one or more individuals who have been monitored.

22. The method according to claim 19, wherein said measured data is collected from at least two blood vessels located respectively on right and left sides of a neck of the individual, the measured data thereby comprising right and left measured data respectively, and the hemodynamic data comprising right and left hemodynamic data respectively.

23. The method according to claim 22, wherein said analyzing of the hemodynamic data comprises analyzing each of the right and left hemodynamic data and applying a comparison between the respective hemodynamic data and a respective baseline data determined based on a first portion of the respective hemodynamic data, to determine the blood supply condition to the brain of the individual.

24. The method according to claim 22, wherein said analyzing of the hemodynamic data comprises applying a comparison between the right and left hemodynamic data to determine the blood supply condition to the brain of the individual.

25. The method according to claim 19, wherein said hemodynamic data comprises one or more of the following: blood vessel's expansion data and blood flow data.

26. The method according to claim 19, wherein said at least one blood vessel is one or more of the following:
common carotid artery, external carotid artery, internal carotid artery and jugular vein.

27. The method according to claim 19, wherein said blood supply to brain condition is indicative of a stroke occurring in the individual's brain.

28. The method according to claim 19, wherein said blood supply to brain condition is indicative of an increase or a decrease of blood supply to the brain.

29. The method according to claim 19, comprising receiving and analyzing medical data in addition to the hemodynamic data for determining the blood supply condition to the brain of the individual.

30. (canceled)
